(19) United States
(12) Patent Application Publication
(10) Pub. No.: US 2019/0183690 A1
(43) Pub. Date: Jun. 20, 2019

(54) ABSORBENT ARTICLES WITH IMPROVED GASKETING

(71) Applicant: KIMBERLY-CLARK WORLDWIDE, INC., Neenah, WI (US)

(72) Inventors: Davis Dang H. Nhan, Menasha, WI (US); Richard N. Dodge, Appleton, WI (US); Peiguang Zhou, Appleton, WI (US); WanDuk Lee, Appleton, WI (US); Thomas A. Eby, Greenville, WI (US); David G. Biggs, New London, WI (US)

(73) Assignee: KIMBERLY-CLARK WORLDWIDE, INC., Neenah, WI (US)

(21) Appl. No.: 16/326,280

(22) PCT Filed: Aug. 31, 2017
(86) PCT No.: PCT/US17/49636

§ 371 Date: Feb. 18, 2019

Related U.S. Application Data
(60) Provisional application No. 62/381,826, filed on Aug. 31, 2016.

(51) Int. Cl. A61F 13/494; A61L 15/22

(52) U.S. Cl. CPC A61F 13/49413; A61F 2013/1591; A61F 2013/4944; A61L 15/225

(57) ABSTRACT

A leg gasket for a disposable absorbent article includes a laminate having a core structure with a first surface and a second surface, the core structure including an elastic core layer and a plastic core layer, wherein the elastic core layer is one of a film, a plurality of strands, and a plurality of strips, wherein the plastic core layer is one of a film layer, a plurality of strands, and a plurality of strips, and wherein at least one of the elastic and plastic core layers is a film; and a nonwoven first facing layer affixed to the first surface. Also, a disposable absorbent article includes a chassis including an absorbent structure and the leg gasket described above.

### BACKGROUND

The present disclosure is generally directed to absorbent articles with composite nonwoven elastic webs. Absorbent product gasketing materials (e.g., flap elastics, leg elastics, and waistband elastics) are indispensable components of absorbent products such as diapers and pants. Gasketing materials are used extensively to complement an absorbent article's absorbent system by serving as barriers to prevent leakage of body fluids from the products. Although the importance of an effective gasketing system is well recognized, there have not been any major efforts focusing on liquid interaction with the gasketing system or on improving the construction and structure of a gasketing system.

A solution to this problem is important because reducing and/or eliminating leakage, especially early leakage, is critical to delivering a consistently positive experience to the user and the caregiver. The present disclosure addresses these issues by providing a composite nonwoven elastic web that includes an elastic web joined to a nonwoven web and processes for forming such composite nonwoven elastic webs. In particular, the present disclosure is directed to elastic laminates and their uses in various product applications.

### SUMMARY

The absorbent products described herein include composites that represent a new class of soft, flexible, and cloth-like nonwoven/film structures that can also potentially be used for a variety of applications such as functional elastics, cleaning wipes, medical fabrics, protection garments, filtration, packaging, and others.

In one aspect, a leg gasket for a disposable absorbent article includes a laminate having a core structure with a first surface and a second surface, the core structure including an elastic core layer and a plastic core layer, wherein the elastic core layer is one of a film, a plurality of strands, and a plurality of strips, wherein the plastic core layer is one of a film layer, a plurality of strands, and a plurality of strips, and wherein at least one of the elastic and plastic core layers is a film; and a nonwoven first facing layer affixed to the first surface.

In an alternate aspect, a disposable absorbent article includes a chassis including an absorbent structure; and a leg gasket attached to the chassis, the leg gasket including an elastic laminate including a core layer having a film layer and a strand or strip, wherein the film layer is one of plastic and elastic and the strand or strip is the other of plastic and elastic, and a nonwoven first facing layer affixed to the core layer.

In another aspect, a method for producing a disposable absorbent article having a leg gasket, the leg gasket including a composite nonwoven elastic web, the method including providing an elastic web comprising a core structure having an elastic core layer and a plastic core layer, wherein the elastic web has a first surface and a second surface; stretching the elastic web to less than 100 percent stretch; affixing a fibrous nonwoven web to the first surface of the stretched elastic web to form a composite nonwoven elastic web; and relaxing the composite nonwoven elastic web. The method also includes activating the composite nonwoven elastic web; forming a leg gasket from the composite nonwoven elastic web; and installing the leg gasket in the disposable absorbent article.

Objects and advantages of the disclosure are set forth below in the following description, or can be learned through practice of the disclosure.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 illustrates an exploded schematic view of an elastic laminate having an elastic layer and a plastic layer in accordance with the present disclosure;

FIG. 2 illustrates an exploded schematic view of an elastic laminate similar to that of FIG. 1, having a plastic layer and an elastic layer in accordance with the present disclosure;

FIG. 3 is a perspective illustration of an example absorbent article incorporating the elastic laminate of FIGS. 1 and/or 2;

FIG. 4A is a schematic cross section of a micro-CT image along the cross direction of the laminate with drawn lines from elastic strands;

FIG. 4B is a schematic cross section of a micro-CT binary image along the cross direction of the laminate with drawn lines from elastic strands;

FIG. 5 is a graphical representation of the results of a micro-CT image analysis;

FIG. 6 is a schematic cross section of micro-CT images of Code 1 (SF4000) along the machine direction of the laminate, taken between elastic strands;

FIG. 7 is a schematic cross section of micro-CT images of Code 1 (SF4000) along the machine direction of the laminate, taken at an elastic strand;

FIG. 8 is a schematic illustration of a GAP testing fixture;

FIG. 9 is a close-up schematic illustration of the GAP testing fixture of FIG. 8 showing a test sample covering the testing slit;

FIG. 10 is a close-up schematic illustration of the GAP testing fixture of FIG. 8 showing securement of the test sample;

FIG. 11 is a close-up schematic illustration of the GAP testing fixture of FIG. 8 showing solution in the GAP testing fixture and a leak in the test sample;

FIG. 12 is a schematic illustration of tensile testing a test sample;

FIG. 13 is a perspective view of the GAP test fixture; and

FIG. 14 is an elevation view of the back side of the GAP test fixture.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

As used herein the term “nonwoven fabric or web” refers to a web having a structure of individual polymeric and/or cellulosic fibers or threads that are interlaid, but not in an identifiable manner as in a knitted fabric. Nonwoven fabrics or webs have been formed from many processes such as for example, meltblowing processes, spunbonding processes, bonded carded web processes, those used to make tissue and towels, etc.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which can be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto. Generally speaking, meltblown fibers can be microfibers that are substantially continuous or discontinuous, generally smaller than 10 microns in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., U.S. Pat. No. 4,340,563 to Appel, et al. and U.S. Pat. No. 5,382,400 to Pike, et al., which are incorporated herein in their entirety by reference hereto thereto. Spunbond fibers are generally not tacky when they are deposited onto a collecting surface. Spunbond fibers can sometimes have diameters less than about 40 microns, and are often between about 5 to about 20 microns.

As used herein the term “staple fiber” means fibers that have a fiber length generally in the range of about 0.5 to about 150 millimeters. Staple fibers can be cellulosic fibers or non-cellulosic fibers. Some examples of suitable non-cellulosic fibers that can be used include, but are not limited to, hydrophilically-treated polyolefin fibers, polyester fibers, nylon fibers, polyvinyl acetate fibers, and mixtures thereof. Hydrophilic treatments can include durable surface treatments and treatments in polymer resins/blends. Cellulosic staple fibers include for example, pulp, thermomechanical pulp, synthetic cellulosic fibers, modified cellulosic fibers, and the like. Cellulosic fibers can be obtained from secondary or recycled sources. Some examples of suitable cellulosic fiber sources include virgin wood fibers, such as thermomechanical, bleached and unbleached softwood and hardwood pulps. Secondary or recycled cellulosic fibers can be obtained from office waste, newsprint, brown paper stock, and paperboard scrap. Further, vegetable fibers, such as abaca, flax, milkweed, cotton, modified cotton, cotton linters, can also be used as the cellulosic fibers. In addition, synthetic cellulosic fibers such as, for example, rayon, viscose rayon, and lyocell can be used. Modified cellulosic fibers are generally composed of derivatives of cellulose formed by substitution of appropriate radicals (e.g., carboxyl, alkyl, acetate, nitrate, etc.) for hydroxyl groups along the carbon chain. Desirable staple fibers for the purposes of this application are hydrophilic, such as traditional cellulosic fibers (a desirable example of which is pulp fibers, as can be found in rolled tissues and paper-based towels).

As used herein, the term “substantially continuous fibers” is intended to mean fibers that have a length that is greater than the length of staple fibers. The term is intended to include fibers that are continuous, such as spunbond fibers, and fibers that are not continuous, but have a defined length greater than about 150 millimeters.

As used herein “bonded carded webs” or “BCW” refers to nonwoven webs formed by carding processes as are known to those skilled in the art and further described, for example, in U.S. Pat. No. 4,488,928 to Ali Khan et al., which is incorporated herein by reference thereto. Briefly, carding processes involve starting with a blend of, for example, staple fibers with bonding fibers or other bonding components in a bulky ball that is combed or otherwise treated to provide a generally uniform basis weight. This web is heated or otherwise treated to activate the adhesive component resulting in an integrated, usually lofty nonwoven material.

The basis weight of nonwoven webs is usually expressed in ounces of material per square yard (osy) or grams per square meter (gsm) and fiber diameters are usually expressed in microns, or in the case of staple fibers, denier. It is noted that to convert from osy to gsm, multiply osy by 33.91.

As used herein, the terms “machine direction” or “MD” generally refers to the direction in which a material is produced. It is also often the direction of travel of the forming surface onto which fibers are deposited during formation of a non-woven web. The term “cross-machine direction” or “CD” refers to the direction perpendicular to the machine direction. Dimensions measured in the cross-machine direction (CD) are referred to as “width” dimensions, while dimensions measured in the machine direction (MD) are referred to as “length” dimensions. The width and length dimensions of a planar sheet make up the X and Y directions of the sheet. The dimension in the depth direction of a planar sheet is also referred to as the Z-direction.

As used herein, the terms “elastomeric” and “elastic” are used interchangeably and shall mean a layer, material, laminate or composite that is generally capable of recovering its shape after deformation when the deforming force is removed. Specifically, when used herein, “elastic” or “elastomeric” is meant to be that property of any material that, upon application of a biasing force, permits the material to be stretchable to a stretched biased length that is at least about fifty (50) percent greater than its relaxed unbiased length, and that will cause the material to recover at least forty (40) percent of its elongation upon release of the stretching force. A hypothetical example that would satisfy this definition of an elastomeric material would be a one (1) inch sample of a material that is elongatable to at least 1.50 inches and that, upon being elongated to 1.50 inches and released, will recover to a length of less than 1.30 inches. Many elastic materials can be stretched by much more than fifty (50) percent of their relaxed length, and many of these will recover to substantially their original relaxed length upon release of the stretching force.

As used herein the term “recover” refers to a contraction of a stretched material upon termination of a biasing force following stretching of the material by application of the biasing force. For example, if a material having a relaxed, unbiased length of one (1) inch was elongated 50 percent by stretching to a length of one and one half (1.5) inches the material would have a stretched length that is 150 percent of its relaxed length. If this exemplary stretched material contracted, that is recovered, to a length of one and one tenth (1.1) inches, after release of the biasing and stretching force, the material would have recovered 80 percent (0.4 inch) of its elongation.

As used herein, the term “g/cc” generally refers to grams per cubic centimeter.

As used herein, the term “hydrophilic” generally refers to fibers or films, or the surfaces of fibers or films that are wettable by aqueous liquids in contact with the fibers. The term “hydrophobic” includes those materials that are not hydrophilic as defined. The phrase “naturally hydrophobic” refers to those materials that are hydrophobic in their chemical composition state without additives or treatments affecting the hydrophobicity.

The degree of wetting of the materials can, in turn, be described in terms of the contact angles and the surface tensions of the liquids and materials involved. Equipment and techniques suitable for measuring the wettability of particular fiber materials or blends of fiber materials can be provided by the Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

As used herein, the term “personal care product” refers to diapers, training pants, absorbent underpants, adult incontinence products, sanitary wipes and feminine hygiene products, such as sanitary napkins, pads, and liners, and the like. The term “absorbent medical product” is employed to refer to products such as medical bandages, tampons intended for medical, dental, surgical, and/or nasal use, surgical drapes and garments, coverings in medical settings, and the like.

The term “composite” as used herein, refers to a film material that has been bonded to or otherwise exists with a nonwoven web including fibers. The film material itself can be mono-layer, multi-component, or multilayer. The composite can be apertured and breathable, or the film material of the composite can be essentially intact.

The present disclosure describes personal care products and absorbent products that incorporate non- or low-stretch bonded (<100% stretch) elastic laminate. Such laminate includes one or two external nonwoven layers and an internal plastic layer adjacent to an internal elastic layer, with adhesive layers between the nonwoven layers and the film/elastic layers. The film and/or elastic layers can be in the form of film, strips, strands, etc. The film can be breathable. The laminate is activated through a groove rolling or intermeshing gears process.

The elastic laminate experiences a nearly-complete fracturing of the nonwoven layers while maintaining the continuity of the plastic layer. This allows the ability to design the stress/strain properties of the laminate while also maintaining the stretch-to-stop requirements for consumer-preferred fit characteristics. This provides a soft, gentle, more underwear-like elastic material for improved leakage performance in personal care garment applications.

Current elastic materials used in products in the global market are generally based on stretch-bonded elastic laminate technology with a nonwoven facing. In the stretch-bonded elastic laminate process, elastic materials (film or strands) are stretched to 3-5 times their relaxed length before being bonded to facing materials.

As a result of the work described herein, it was found, for example, that non-stretch bonded elastic strands combined with a thin/strong plastic film and facing materials can provide superior performance compared to current stretch-bonded elastic laminate materials. Furthermore, the non-stretch bonded elastic laminate of this invention also unexpectedly creates desirable surface texture and appearance that can provide a more consumer-preferred appearance in terms of lighter, softer, gentler, and more cloth-like as underwear.

More specifically, the elastic laminate of the present disclosure can include either an elastic strand and a plastic film non-stretch bonded to a nonwoven, an elastic film and a plastic strand non-stretch bonded to a nonwoven, or any elastic material form and any plastic material form non-stretch bonded to a nonwoven. The elastic and plastic layers can be in the form of film, strips, strands, etc. The laminate is activated through a groove rolling process. This elastic laminate provides consumer-preferred appearance and strength, as well as designable Stretch-to-Stop (S-S) curves.

The groove rolling process results in the nearly-complete fracturing of the nonwoven layers while maintaining the continuity of the plastic layer. This allows the ability to design the stress/strain properties of the laminate while also maintaining the stretch-to-stop requirements for consumer-preferred fit characteristics.

The main purpose in flap gasketing is ensuring a good seal between the flap elastic and the surface (skin of the wearer). Creating this seal between the flap elastic and surface can be accomplished through two primary mechanisms. The first mechanism is using a flap that has topography with less (smaller) gapping when placed in contact with the surface. The second is to use force from the elastic strands to collapse the gapping caused by the structures.

A typical elastic laminate (also known as Lycra laminate, LAWN) for flap applications consists of a facing (SMS), two elastic strands, and a hot melt adhesive. The laminate is made by stretching the elastic strands to a specific elongation (typically 260-270%); the stretched elastic strands are then adhesively bonded to the facing material.

The elastic laminate described herein provides an improved gasket construction for absorbent articles. The improved gasket is a non-stretch bonded strand/film elastic laminate (NSBSFEL) material such as that described in Patent Application No. PCT/US16/20017 filed on Feb. 29, 2016, and that is incorporated herein by reference to the extent it does not conflict herewith. The present disclosure provides a better leg gasket than prior art gaskets through the minimization of micro-gaps between the gasketing material and the wearer's body.

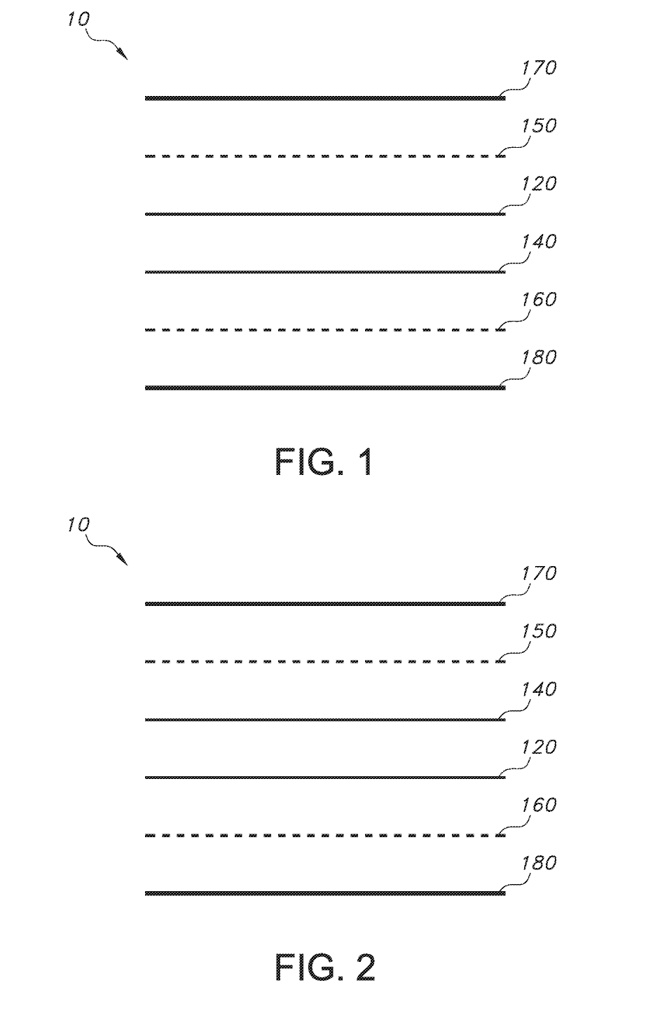

In a particular aspect of the present disclosure illustrated in FIG. 1, an elastic laminate 10 includes a core structure having a first surface and a second surface, the core structure including an elastic core layer 120 and a plastic core layer 140. The elastic core layer 120 is one of a film, a plurality of strands, and a plurality of strips. The plastic core layer 140 is also one of a film layer, a plurality of strands, and a plurality of strips. The elastic core layer 120 and the plastic core layer 140 can be the same format or of different formats. The plastic core layer 140 reinforces the core structure in the machine direction. The elastic laminate 10 also includes two low basis weight (˜8 gsm) facing materials 170, 180 affixed to the core structure by any suitable means. In one aspect of the present application, the facing materials 170, 180 are affixed to the core structure with adhesive bonding layers 150, 160. In other aspects, the elastic laminate 10 can be manufactured without one or both facing materials 170, 180.

In an alternate aspect of the present disclosure illustrated in FIG. 2, an elastic laminate 10 includes a core structure having a first surface and a second surface, the core structure including an elastic core layer 120 and a plastic core layer 140. The elastic core layer 120 is one of a film, a plurality of strands, and a plurality of strips. The plastic core layer 140 is also one of a film layer, a plurality of strands, and a plurality of strips. The elastic core layer 120 and the plastic core layer 140 can be the same format or of different formats. The plastic core layer 140 reinforces the core structure in the machine direction. The elastic laminate 10 also includes two low basis weight (˜8 gsm) facing materials 170, 180 affixed to the core structure by any suitable means. In one aspect of the present application, the facing materials 170, 180 are affixed to the core structure with adhesive bonding layers 150, 160. In other aspects, the elastic laminate 10 can be manufactured without one or both facing materials 170, 180.

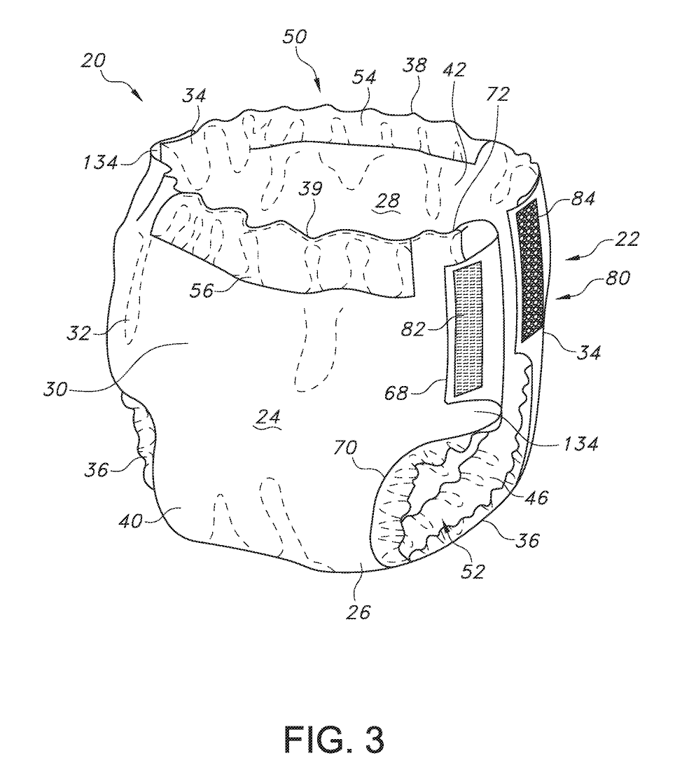

Returning to the aspect illustrated in FIG. 1, the elastic core layer 120 is adhesively bonded to the plastic core layer 140 and facing materials 170, 180 under little or no elongation, such as less than or equal to 100%. After the elastic laminate 10 is made, it has a very little elasticity and the laminate 10 is not an elastic material at this point (maximum elongation is less than 20% with minimum tension). The laminate 10 is then stretched (200-300%) in the machine direction (MD) using a groove rolling technique, an intermeshing gears process, or any other suitable process. This stretching locally tears the facing materials 170, 180, extends the plastic core layer 140 up to 20-300% depending on processing conditions, and turns the laminate 10 into a very elastic material with up to 150-250% elongation and with designable tension and S-S curves as shown in FIG. 3, and as described in more detail below.

The elastic laminate 10 in this aspect permanently exhibits a fine regular and periodic three-dimensional structure even under maximum stretching conditions. In contrast, current stretch-bonded elastic laminates demonstrate a generally flat surface when under maximum stretching conditions. The fine three-dimensional surface structure of the laminate 10 provides a more consumer-preferred premier appearance and softer, gentler feel. In this aspect, the plastic core layer 140 functions to provide strength and a stretch-to-stop property to the elastic laminate 10. The elastic core layer 120 contributes elastic performance for seal, fit, and comfort functions when employed in a personal care article. The facing materials 170, 180 provide a cloth-like appearance to the elastic laminate 10.

The film can be either plastic or elastic. One example of a suitable film includes a high strength and/or extendable plastic film, whether single or multilayer, incorporated into the laminate 10 to provide a designable tear/poke through strength as well controllable stretch-to-stop curves for product application and consumer prefer appearance attributes. Other examples of suitable films include but are not limited to wrap polyfilm, both polypropylene (PP)- and polyethylene (PE)-based, breathable outer cover film, organoclay nanocomposite film, and elastic film. In a particular exemplary aspect, the film has a thickness of 0.2-2 mil, a basis weight 10-30 gsm, an elongation of 100-600%, a tensile modulus (D638) of 40-200 m/pa, and a Shore D hardness of 30-45, where the preferred polymers include linear low-density polyethylene (LLDPE), low-density polyethylene (LDPE), and polyolefin copolymers.

The strands and strips can also be plastic or elastic. One example of a suitable strand material can be a pre-made strand or a strand extruded from thermoplastic elastomers to provide great elastic performance. Pre-made strands include LYCRA-brand elastic strands available from INVISTA, and CREORA-brand elastic fibers available from Hyosung. Another example of suitable strands is an extrudable strand including thermoplastic polyurethane (TPU) available from Huntsman. Other examples of suitable strands include but are not limited to polypropylene-based thermo elastomers such as VISTAMAXX-brand elastomer polymers available from ExxonMobil Chemical, styrenic block copolymer (SBC) such as KRATON-brand SBC available from Kraton Performance Polymers, and olefin block copolymers (OBC) such as INFUSE-brand olefin block copolymers available from Dow Chemical. Similarly, plastic strands can be either pre-made or extruded from thermoplastic polymers.

The facing materials can be any suitable material including nonwovens such as tissue, spunbond, meltblown, or any other suitable cellulose- or polymer-based material. Low basis weight and/or low strength nonwovens can provide a cloth-like appearance with softer and gentler touch after activation. All components can be adhesively laminated or extrusion laminated.

The adhesive employed to bond the facing materials to the elastic/plastic layers can be any suitable adhesive.

In other aspects of the present disclosure, the non-stretch bonding of the layers of the elastic laminate 10 can be also accomplished by any other suitable method including, but not limited to, thermal, ultrasonic, and extrusion lamination bonding.

Ring rolling is well known in the art. Examples of descriptions of the process include those in patent application EP650714 to Coles et al.

Non-stretch bonded strand/film elastic laminates as described herein demonstrate performance enhancements including premier appearance, gentle and soft feel and touch, increased strength through strand enforcement, controllable stretch-to-stop curves (100-250%), and breathable but liquid impermeable performance. In addition, the elastic laminate 10 described herein demonstrates a cost that is less than current elastic laminates because the elastic laminate 10 has a basis weight that can be reduced to up to 50 percent of the basis weight of current elastic laminates made from stretch-bonded laminate processes.

The materials that can be used to form the fibrous nonwoven web or facing materials 170, 180 include any nonwoven material capable of performing as described above. For example, the facing materials 170, 180 can be formed from a blend of a non-elastic material with an elastic material, one or more non-elastic materials or a blend of one or more elastic materials with two or more non-elastic materials. Preferably, the facing materials 170, 180 are formed from a fiber-forming meltblowable or spunbondable non-elastic gatherable material. However, the facing materials 170, 180 can be formed by depositing a carded web on the surface of the core structure or by any other method which may be utilized to form facing materials 170, 180 on the surface of the core structure. Exemplary fiber-forming materials for use in forming the facing materials 170, 180 are polyester materials, polyolefin materials or blends of one or more polyester materials with one or more polyolefin materials. An exemplary polyester fiber-forming material is polyethylene terephthalate. An exemplary fiber-forming polyolefin material is polypropylene. Preferred polypropylene materials, for example, can be obtained from the Himont Company under the trade designations PC 973 and PF 301.

After the facing materials 170, 180 have been formed upon or affixed to the upper surface of the core structure, the composite nonwoven elastic web 10 is passed through rollers that, for the reasons stated above, need not be heated or need not apply any excessive pressure to the core structure. Thereafter, the stretching and biasing force on the core structure is released so as to relax and contract the composite nonwoven elastic web 10.

Preferably, the elastic films used in the present invention can stretch at least 50%-100% in comparison to its non-stretched length. In some cases, it is desirable that films can be stretched up to 100%-200%, 200%-400%, or 400%-600%.

An exemplary use of the composite nonwoven elastic web 10 employs the composite nonwoven elastic web 10 in a leg gasket in the form of a containment flap in a pair of training pants 20, as representatively illustrated in FIG. 3 in a partially fastened condition. The leg gasket can be a containment flap or any other suitable structure intended to inhibit or eliminate leakage from the absorbent article.

The pants 20 define a pair of longitudinal end regions, otherwise referred to herein as a front region 22 and a back region 24, and a center region, otherwise referred to herein as a crotch region 26, extending longitudinally between and interconnecting the front and back regions 22, 24. The pant 20 also defines an inner surface 28 adapted in use (e.g., positioned relative to the other components of the pants 20) to be disposed toward the wearer, and an outer surface 30 opposite the inner surface. The front and back regions 22, 24 are those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The training pants 20 have a pair of laterally opposite side edges 36 and a pair of longitudinally opposite waist edges, respectively designated front waist edge 38 and back waist edge 39.

The illustrated pants 20 include a chassis 32, a pair of laterally opposite front side panels 34 extending laterally outward at the front region 22 and a pair of laterally opposite back side panels 134 extending laterally outward at the back region 24.

Referring to FIG. 3, the chassis 32 includes an outer cover 40 and a bodyside liner 42 that may be joined to the outer cover 40 in a superimposed relation therewith by adhesives, ultrasonic bonds, thermal bonds or other conventional techniques. The liner 42 may suitably be joined to the outer cover 40 along the perimeter of the chassis 32 to form a front waist seam 62 and a back waist seam 64. The liner 42 can be generally adapted, i.e., positioned relative to the other components of the pants 20, to be disposed toward the wearer's skin during wear of the pants. The chassis 32 may further include an absorbent structure 44 disposed between the outer cover 40 and the bodyside liner 42 for absorbing liquid body exudates exuded by the wearer, and may further include a pair of containment flaps 46 secured to the bodyside liner 42 for inhibiting the lateral flow of body exudates.

With the training pants 20 in the fastened position as partially illustrated in FIG. 3, the front and back side panels 34, 134 can be connected together by a fastening system 80 to define a three-dimensional pants configuration having a waist opening 50 and a pair of leg openings 52. The front and back side panels 34 and 134, upon wearing of the pants 20, thus include the portions of the training pants 20 which are positioned on the hips of the wearer. The waist edges 38 and 39 of the training pants 20 with their front and back waist elastics 56, 54 are configured to encircle the waist of the wearer and together define a waist opening 50 of the pants.

The elasticized containment flaps 46 as shown in FIG. 3 define a partially unattached edge which assumes an upright configuration in at least the crotch region 26 of the training pants 20 to form a seal against the wearer's body. The containment flaps 46 can extend longitudinally along the entire length of the chassis 32 or may extend only partially along the length of the chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

Reference now will be made in detail to various aspects of the disclosure, one or more examples of which are set forth below. Each example is provided by way of explanation, not of limitation of the disclosure. In fact, it will be apparent to those skilled in the art that various modifications and variations can be made in the present disclosure without departing from the scope or spirit of the disclosure. For instance, features illustrated or described as part of one aspect, can be used on another aspect to yield a still further aspect. Thus it is intended that the present disclosure cover such modifications and variations.

The sheet materials produced in accordance with this disclosure can be used in a variety of end product applications. It is contemplated that such sheet materials have end product applications including in the technical areas of filtration, medical garments, covers, and bandages, and the personal care area, such as in the ears or side panels of baby/child care diapers, and adult feminine care applications. Articles made under this disclosure are very flexible and soft with a cloth-like feel.

In one mode of gasketing failure, liquid penetrates the interface between the gasketing material and the wearer's body through “micro-gaps.” These micro-gaps typically are not visible to the naked eye, and can range from tens to hundreds of microns. These micro-gaps arise due to the micro-fold topography of facing material as elastic members retract. Non-stretch bonded strand film elastic laminate (NSBSFEL) material was tested for its ability to minimize and/or eliminate micro-gaps.

A Micro-CT test method is used to develop an understanding of what features of the flap impact contact at the flap-human body interface and to quantify these structures to find the correlation between flap structure and gasketing ability. The micro-CT imaging technique provided a 3-D view of the interface between a flap elastic material and surface. It also provided strand-to-skin distance data that is important to measure the gasketing ability of flap elastic materials.

Method to Measure Distance Between Gasket (Flap) and Simulated Body Surface

The distance between the gasket structure and the opposing simulated body surface can be determined by using the x-ray Micro-computed tomography (a.k.a. Micro-CT) and image analysis measurement methods described herein. In this context, distance is considered between the gasket's elastomeric strand nearest its surface that opposes the wearer's body surface. Generally, the image analysis method determines a numeric value of linear distance between the gasket surface opposing a simulated body surface. The gap distance method is performed using Micro-CT to non-destructively acquire images with subsequent image analysis techniques to perform the distance measurements. To perform the distance measurement, an image analysis algorithm is used that includes specific image processing and measurement steps. The image analysis algorithm performs detection, image processing, and measurement and also transmits data digitally to a spreadsheet database. The resulting measurement data are used to compare the gap distance of differing gasket structures.

The method for determining the gap distance between the gasket and opposing simulated body surface includes the first step of acquiring digital x-ray Micro-CT images of a sample. These images are acquired using a SkyScan 1272 Micro-CT system available from Bruker microCT (2550 Kontich, Belgium). Prior to mounting the sample in the Micro-CT, the gasket was attached to a piece of closed-cell coating-free extruded polystyrene foam (taken from a circle with a diameter of 6″ to simulate the same curvature as the GAP test) at approximately 60% elongation using adhesive tape. The foam used was a smooth foam disc with dimensions of 22 mm×149 mm, available from FloraCraft Corporation in Ludington, Mich. The elastic/closed-cell extruded polystyrene foam assembly was then trimmed to fit on a sample spindle to minimize the amount of unnecessary closed-cell extruded polystyrene foam. The gasket and simulated body surface (closed-cell extruded polystyrene foam) sample is then attached to a mounting apparatus, supplied by Bruker with the SkyScan 1272 system, so that it will not move under its own weight during the scanning process. Also, oversize scanning was done to encompass the entire sample in the vertical direction, and double wide scanning to encompass the entire width. This results in a 3 high×2 wide field (6 individual scans) that is computer-assembled into the final seamless mosaic. The following SkyScan 1272 conditions are used during the scanning process:

Camera Pixel Size (um)=9.0

Source Voltage (kV)=40

Source Current (uA)=250

Image Pixel Size (um)=19.6

Image Format=TIFF

Depth (bits)=16

Rotation Step (deg.)=0.40

Use 360 Rotation=NO

Frame Averaging=ON (3)

Random Movement=ON (2)

Flat Field Correction=ON

Filter=No Filter

After sample scanning is completed, the resulting image set needs to be reconstructed using the NRecon program provided with the SkyScan 1272 Micro-CT system. While reconstruction parameters can be somewhat sample dependent, and should be known to those skilled in the art, the following parameters should provide a basic guideline to an analyst:

Image File Type=BMP

Pixel Size (um)=19.60

Smoothing=0

Ring Artifact Correction=12

Beam Hardening Correction (%)=20

After reconstruction is completed, the resulting image data set is now ready for image analysis.

The image analysis software platform used to perform the linear distance measurements is QWIN Pro (Version 3.5.1) available from Leica Microsystems, having an office in Heerbrugg, Switzerland.

Thus, the method for determining the gap distance of a given specimen includes the step of performing one to two distance measurements on the Micro-CT image. Specifically, an image analysis algorithm is used to read and process images as well as perform measurements using Quantimet User Interactive Programming System (QUIPS) language. The image analysis algorithm is reproduced below.

NAME = Distance Between Objects − 1
PURPOSE = Measures linear distance between one or more
objects (semi-manual)
CONDITIONS = Various image sources (e.g. optical, micro-CT,
SEM, etc.)

The following line designates the computer location where data is sent to

Open File (D:\Data\xxxxxx.xls)
Set-Up
Enter Results Header
File Results Header (channel #1)
File Line (channel #1)

The following two lines are based on the size of the reconstructed Micro-CT images

Image frame (x 0, y 0, Width 432, Height 304)
Measure frame (x 23, y 19, Width 386, Height 272)
-- Calibration = 19.6 um/px
CALVALUE = 19.6
Calibration (Local)
For (SAMPLE = 1 to 1, step 1)

The following line is based on the image file prefix from the Micro-CT image set being analyzed.

PauseText (“Enter image prefix now.”)
Input (TITLE$)

The following line is based on the image file suffix numbers from the Micro-CT image set being analyzed. For the image set shown below, every 25^{th} image was sampled.

For (REPLICATE = 1000 to 1550, step 25)
IMAGE ACQUIRE
Binary Edit (Clear Binary5)
ACQOUTPUT = 0

The following two lines indicate the computer location of the Micro-CT images to be read during the image analysis process.

ACQFILE$=“D:\Images\xxxxxx\
“+TITLE$+”“+STR$(REPLICATE)+”.bmp”
Read image (from file ACQFILE$ into ACQOUTPUT)
Colour Transform (Mono Mode)
Grey Transform (WSharpen from Image0 to Image1, cycles 2,
operator Octagon)
Display (Image0 (on), frames (on,on), planes (off,off,off,off,off,off),
lut 0, x 0, y 0, z 1, Reduction off)
Detection & Processing

The following line is the gray-level threshold level for detecting the gasket material and closed-cell extruded polystyrene foam simulated body surface components within an image. The threshold may need to be adjusted prior to executing the algorithm to reflect optimal detection.

Detect (whiter than 110, from Image1 into Binary0)
Binary Amend (Open from Binary0 to Binary1, cycles 2, operator
Disc, edge erode on)
Binary Identify (FillHoles from Binary1 to Binary2)
Binary Amend (Close from Binary2 to Binary3, cycles 6, operator
Disc, edge erode on)
PauseText (“Draw lines between items of interest.”)
Binary Edit [PAUSE] (Vector from 161,78 to 162,109, width 2, in
Binary4)
Binary Logical (C = A XOR B: C Binary5, A Binary3, B Binary4)
Measurements
Clear Accepts
Measure feature (plane Binary5, 8 ferets, minimum area: 8,
grey image: Image0)
Selected parameters: X FCP, Y FCP, Feret90, Length
File Feature Results (channel #1)
Display Feature Results (x 1346, y 613, w 568, h 404)
Next (REPLICATE)
Next (SAMPLE)
END

The QUIPS algorithm is executed using the QWIN Pro software platform. The analyst is initially prompted to enter the specimen set information which is sent to the EXCEL file.

The analyst is next prompted by an interactive command window and an input window to enter the image file prefix of the Micro-CT images to be analyzed. After this step, all subsequent images for a given sample will be read for analysis automatically by the algorithm Distance Between Objects−1.

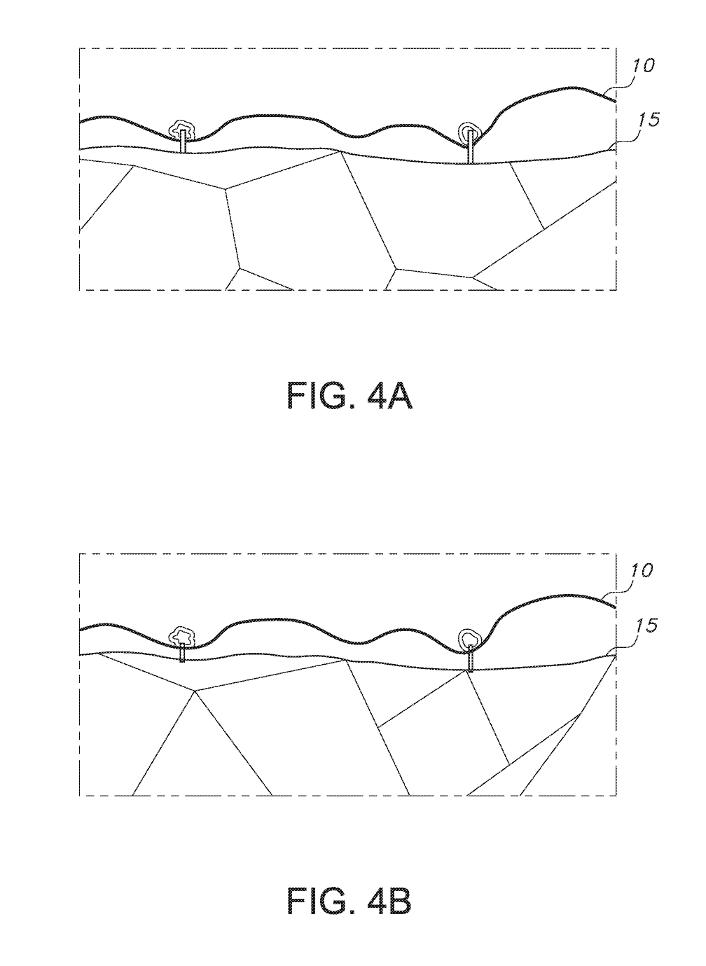

The analyst is next prompted to manually place a line between the center of the now detected bright circular elastic strands and the top surface of the closed-cell extruded polystyrene foam simulated body surface of the sample cross-section as viewed in the image window. This is performed by using the computer mouse to draw a straight line between these two items of interest (see FIG. 4A).

After a single line has been drawn between elastomeric strand centers and closed-cell extruded polystyrene foam surface 15 just below, the analyst then continues the algorithm by clicking on the ‘OK’ or ‘Continue’ buttons shown on the screen. A similar prompting will occur for all subsequent images automatically read by the algorithm.

After the algorithm automatically performs a binary image processing step to isolate the distance between the bottom edge of the elastic strand and top edge of the closed-cell extruded polystyrene foam surface 15, the final binary is automatically measured for its length and the data is exported to the designated EXCEL spreadsheet file (see an example of a final binary image, FIG. 4B). The algorithm will then automatically read the next image and prompt the analyst to again repeat the process described in the previous paragraph. This process is repeated until all of the designated images have been analyzed.

The length measurement parameter data will be located in the EXCEL file after measurements and data transfer has occurred. The length measurement can be considered the distance measurement from which statistics such as average and standard deviation can be calculated.

Multiple sampling replicates from a single specimen can be performed during a single execution of the QUIPS algorithm from reading in multiple images (Note: The REPLICATE For-Next line in the algorithm needs to be adjusted to reflect the number of sample replicate analyses to be performed per specimen). For example, from a Micro-CT image set of 1000 images, every 20^{th} image can be analyzed resulting in 50 data points per sample. A comparison between different samples can be performed using a Student's T analysis at the 90% confidence level.

### GAP Test Method

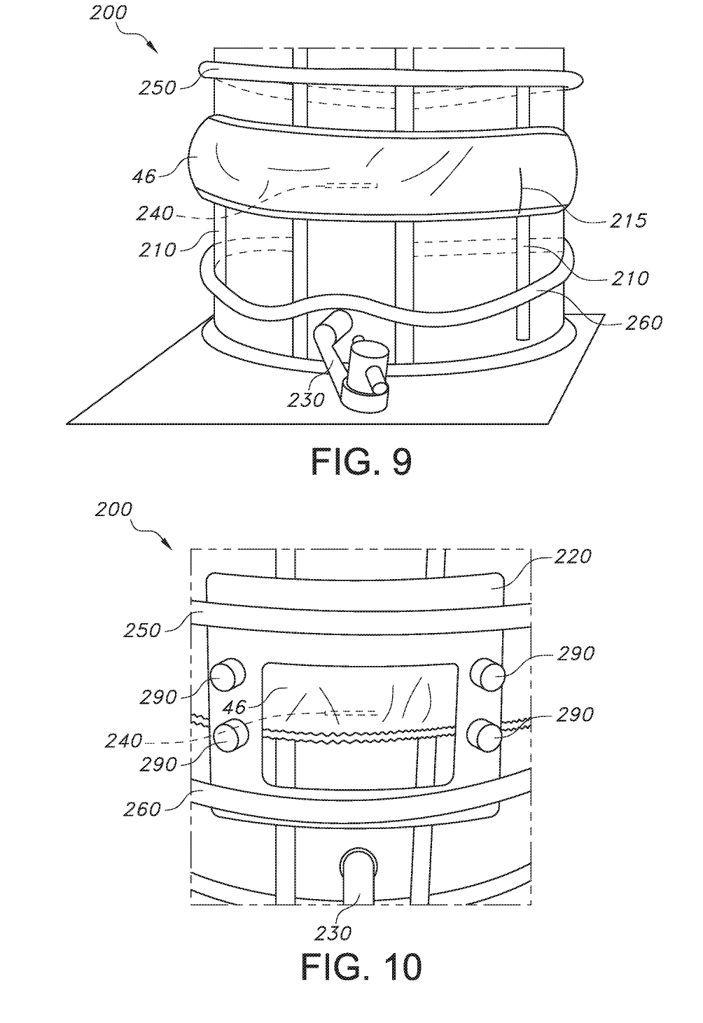

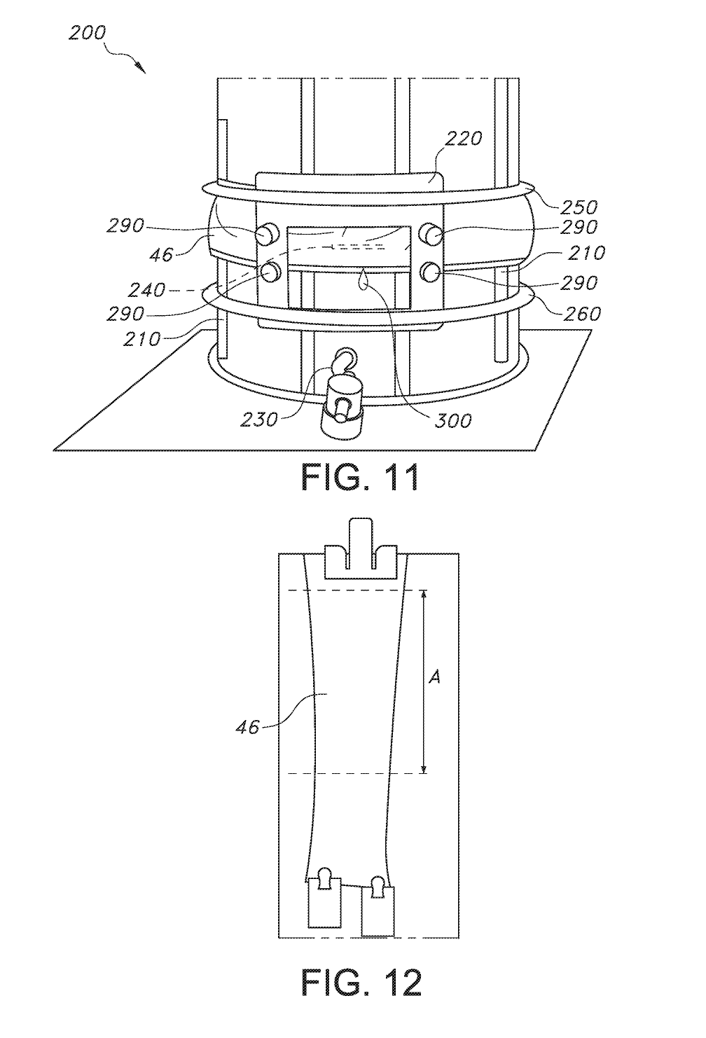

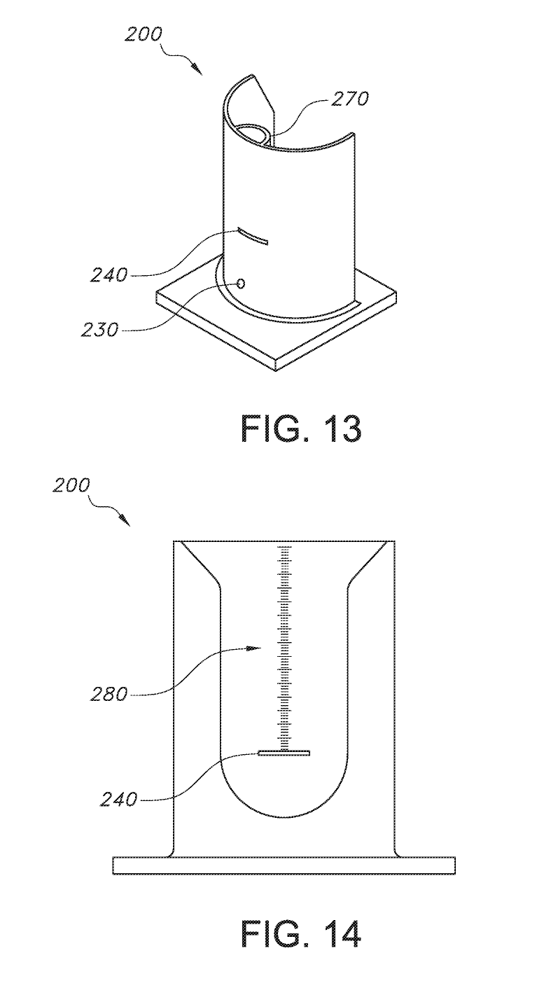

The fixture 200 used in GAP testing is illustrated schematically in FIGS. 8-11, 13, and 14. The GAP test fixture 200 was produced by Midwest Prototyping, LLC, made of Accura 60 material with a level 3 finish, where the level indicates the level of sanding after applying a polyurethane coating. The GAP test fixture 200 is designed to test the ability of a flap material to limit or prevent leakage past the seal produced by a flap 46.
1. Ensure that equipment is properly set-up, see main points below:
   a. Ensure that GAP test fixture 200 (see FIGS. 8-11, 13, and 14) is clear of any previous testing solutions, the surface is wiped off so that it is not wet, the hook strips 210 are clear of any excess debris, the attachment piece 220 has no cuts, scrapes, or other defects.
   b. Ensure that tubing is clear of solution and is properly connected to the three-way valve and the GAP test fixture 200 itself at the hose connection 230.
   c. The setting of the pump is at a rate of 75 mL/min
   d. For GAP heights expected below 15 mm, more accurate results can be obtained by stopping the solution every 2-3 mm for a few seconds to ensure that the gasket is holding).
2. With the equipment properly set-up, take a flap 46 that was previously marked per “Procedure for Preparing Flap Samples” and align the flap 46 such that the elastic strands are both below the slit 240 in the test fixture 200 so that the remaining facing covers the slit 240 in the GAP test fixture 200 (see FIG. 9). For the folded portion of the flap 46, testing was performed with the fold touching the test fixture 200.
3. With the flap 46 in the proper orientation, ensure that the marks 215 on the flap 46 are in position with the sides of the hook strips 210 that are closer to the slit 240 (see FIG. 9). The flap 46 is aligned with the hook strips 210 in this procedure because the distance between the inside sides of the hook strips 210 was measured and used for reference in calculating elongation (B=16 cm). Also ensure that the bottommost strand of elastic relative to the slit 240 is placed in the same location each time by aligning this strand with the marks on the test fixture 200. Marks were made on the test fixture 200 to ensure that the flaps were all placed in the same location each time. These marks were 1.75 cm below the slit 240 and at the same relative height next to each hook strip 210.
4. After the flap 46 is properly aligned, press the facing onto the hook strips 210 to hold it in place. Repeat this step for both sides of the flap 46.
5. Once both sides are secured, ensure that none of the facing is folded over itself and that the flap 46 is level.
6. Next take the attachment piece 220 and set it so that the bottom of the attachment piece 220 is sitting on the platform pieces before gently allowing the attachment piece 220 to press down on the flap 46, at which point it should be held in place by magnets 290 (see FIGS. 10 and 11).
7. Taking the top O-ring 250, lift and place in the groove on the attachment piece 220, ensuring that the attachment piece 220 does not move (see FIG. 10). Repeat this process with the bottom O-ring 260.
8. Once the flap 46 has been properly placed and the attachment piece 220 successfully placed and sealed, begin filling the solution column 270 (see FIG. 13) of the GAP test fixture 200 with the desired solution such as that described below.
9. Continue filling the test fixture 200 with solution until the flap 46 begins to leak (see FIG. 11: Note the leak 300 past the bottom of the flap 46). At the occurrence of a leak, stop the pump and immediately check the height of the solution in the solution column 270 using the ruler 280 (see FIG. 14) on the test fixture 200.
10. Record this data as well as any observations from the test (such as failure mode or location of leak) and empty the solution from the test fixture 200.
11. After drained below the slit 240, remove the attachment piece 220 and the tested flap 46. Wipe off the test fixture 200 as well as the attachment piece 220 until both are completely dry.
12. Repeat until all samples have been tested.

### 52 Dyne Testing Solution

Obtain 3 liters of 0.9% (w/w) saline solution and place in pitcher.
Measure out 3 grams of Pluronic F-38 and add to the saline solution.
Using a magnetic stirrer, begin mixing at a medium speed.
Add 5 pipetfuls of blue dye solution (1 gram of FD&C Blue No. 1 Powder in 1 liter DI water)
Stir for an additional 30 minutes on medium speed.

Procedure for Preparing Samples for GAP Testing
1. A sample is attached at the top of a measuring board with a clip (see FIG. 12).
2. 500 grams is attached to the bottom of the elastic.
3. The elastic is allowed to hang freely in the vertical direction. The flap 46 should be fully extended straight up and down.
   a. The flap 46 is now hanging vertically against the measuring board. The measuring board has marks at a set length (A) which correspond to different elongation percentages on the GAP Test fixture 200.
   b. A is calculated by: A=100%×B/E
      Where the variables are:
         B is the target length to relax to from A to achieve E% elongation. B is also the distance between the two hook strips 210 on the GAP test fixture 200, which is 16 cm
         A is the length of the flap 46 at 100% elongation (fully stretched out)
         E is the percent elongation of the flap 46. This elongation is achieved when the flap 46 is relaxed from A to B
      For example: At E=60% flap elongation and B=16 cm, them A=26.67 cm
      For example: At E=100% flap elongation and B=16 cm, then A=16 cm
4. Wherever the flap 46 is touching the marked locations on the measuring board, make marks on the flap 46.
5. After marking the flap 46, remove the weight at the bottom and unclip the sample from the measuring board. Repeat for as many samples as needed.
   Table 1 shows a brief description of the codes that were analyzed, where Code 2 is the control.

TABLE 1
Material Compositions

| | Facing | Elastic | Hotmelt | | Facing |
|---|---|---|---|---|---|
| Code | material 1 | strands | Adhesive | Plastic film | material 2 |
| 1 | 8 gsm | Creora 620 | Bostik | Clear 0.6 | 8 gsm |
| | meltblown | Dtex | H4258 | mil Stretch | meltblown |
| | | | | wrap film | |
| 2 | 15 gsm | Lycra 800 | Bostik | | 15 gsm |
| | SMS | Dtex | H4258 | | SMS |
| 3 | 8 gsm | Lycra 800 | Bostik | | 8 gsm |
| | SMS | Dtex | H4258 | | SMS |

Bostik H4258 is available from Bostik, Inc., located in Wauwatosa, Wisconsin.

Creora 620 Dtex from Hyosung Holdings USA, Inc., located in Charlotte, N.C.
Lycra 800 Dtex from Invista, located in Wichita, Kans.
8 gsm SMS (spunbond-meltblown-spunbond) available from Avgol America, located in Mocksville, N.C.
15 gsm SMS (spunbond-meltblown-spunbond) available from Avgol America, located in

Mocksville, N.C.
Clear 0.6 mil (0.0006″) stretch wrap film (catalog number: 2008T21) available from Mcmaster-carr, located in Elmhurst, Ill.
8 gsm meltblown material was made from polypropylene purchased from LyondellBasell under the trade designation MF650W.

Code 1: The first laminate was SF4000 material, a non-stretch bonded strand film elastic laminate (NSBSFEL) material. This elastic material was constructed by using 8 gsm meltblown facing material, Creora 620, stretched to ˜50% elongation, 0.6 mil clear stretch-wrap film, and H4258 adhesive with total add-on of approximately 8gsm. The laminate was then machine-direction grooved using intermeshing rolls at 0.27 inch engagement depth (equivalent to ˜325% stretching). The flap 46 was then attached to a piece of closed-cell extruded polystyrene foam (taken from a circle with a diameter of 6″ to simulate the same curvature as the GAP test) at 60% elongation, as described above. Table 2 shows the conditions under which the material was processed.

TABLE 2
Elastic process conditions

| | | | Percent elongation |
|---|---|---|---|
| | Elastic | Machine | of laminate |
| | elongation | direction | as attached on |
| | during | stretching after | the foam for |
| Code | lamination | lamination | micro-CT imaging |
| 1 | 50% | Engagement | 60% |
| | | depth = 0.27″ | |
| 2 | 275% | | 60% |
| 3 | 275% | | 60% |

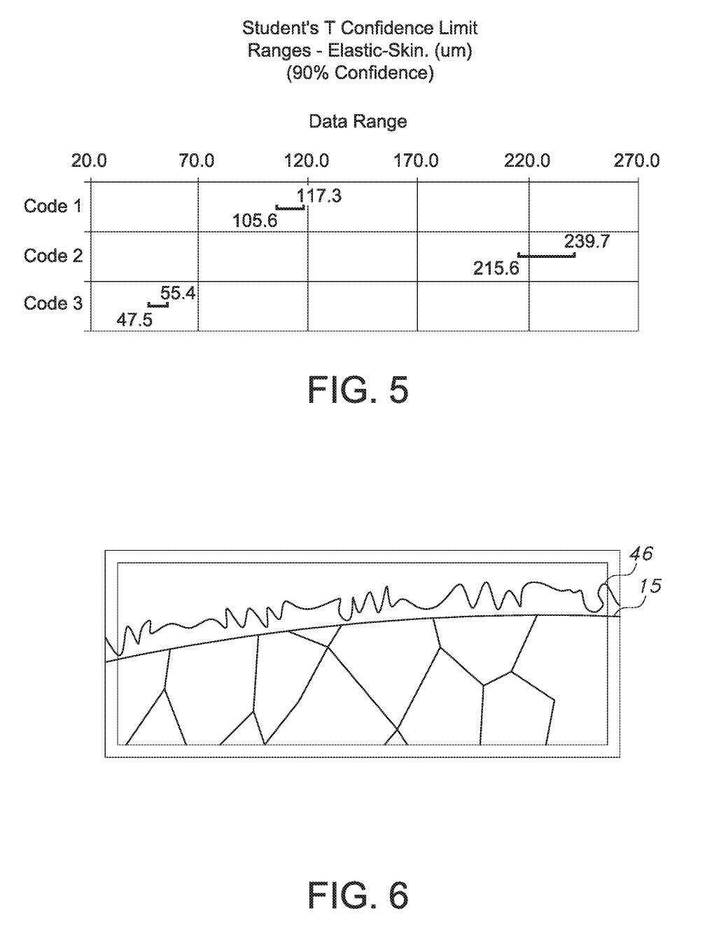

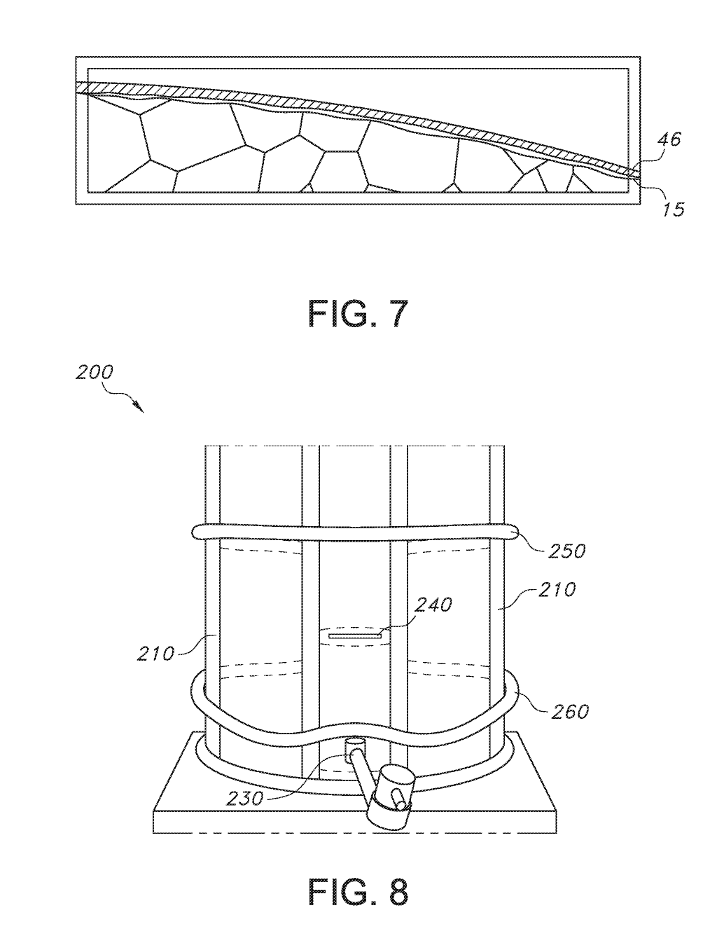

FIG. 5 shows how the distances from the elastic strands to the surface for Codes 1-3. The listed distances are in units of micrometers (microns). The results for Code 1 SF4000 are illustrated in FIGS. 6 and 7 for a schematic cross section of micro-CT images of Code 1 SF4000 along the machine direction of the laminate, taken between elastic strands (FIG. 6) and for a schematic cross section of micro-CT images of Code 1 SF4000 along the machine direction of the laminate, taken at an elastic strand (FIG. 7). When compared to standard materials at a similar 60% elongation, the Code 1 SF4000 has a much smaller gap between the strands and the surface. The gap result is unexpected because the SF4000 was constructed with 620 Dtex strands, and has a lower tensile loading relative to standard materials with heavier strands. Furthermore, SF4000 has similar tensile loading as compared to standard materials with lighter strands. SF4000 still has a much smaller gap as compared to these standard materials. This result shows that the structure of a flap 46 is more important in reducing the distance from flap 46 to surface than by increasing the force. This also means that it is possible to create a lower tension flap elastic material with better gasket by using a laminate that has a much smaller gap structure.

### Results

As shown in Table 3, testing of the materials described herein demonstrated that Code 1 material had significantly better GAP test results. The GAP test method essentially reports a height of a water column before leakage occurs of a gasket stretched to 60% elongation, where a higher water column height indicates less leakage. GAP indicates much better results with the Code 1 material (18 mm) than for prior art Code 2 15 gsm SMS flaps (0 mm).

Micro-CT analysis with an image analysis algorithm to measure the distance from the elastic strand to the surface at 60% elongation (which is representative of the micro-gap) was developed, where a smaller distance indicates reduced micro-gaps. This testing showed that the Code 1 material had the best performance (228 to 254 microns), well ahead of prior art 15 gsm SMS flaps (467 to 519 microns).

TABLE 3
Data

| | | Micro-gap data: Elastic |
|---|---|---|
| Code | GAP result | Strand to Foam Distance |
| 1 | 18 mm | 242 micron |
| 2 | 0 mm | 494 micron |
| 3 | 9.6 mm | 303 micron |

In a first particular aspect, a leg gasket for a disposable absorbent article includes a laminate having a core structure with a first surface and a second surface, the core structure comprising an elastic core layer and a plastic core layer, wherein the elastic core layer is one of a film, a plurality of strands, and a plurality of strips, wherein the plastic core layer is one of a film layer, a plurality of strands, and a plurality of strips, and wherein at least one of the elastic and plastic core layers is a film; and a nonwoven first facing layer affixed to the first surface.

A second particular aspect includes the first particular aspect, wherein the plurality of strands is disposed between the film layer and the first facing layer.

A third particular aspect includes the first and/or second aspect, wherein the first facing layer is affixed to the first surface with adhesive.

A fourth particular aspect includes one or more of aspects 1-3, further including a nonwoven second facing layer affixed to the second surface.

A fifth particular aspect includes one or more of aspects 1-4, wherein the nonwoven first facing layer is cellulose-based.

A sixth particular aspect includes one or more of aspects 1-5, wherein the nonwoven first facing layer is polymer-based.

A seventh particular aspect includes one or more of aspects 1-6, wherein the nonwoven first facing layer includes polymer and cellulose.

An eighth particular aspect includes one or more of aspects 1-7, wherein the plastic core layer is a film, and wherein the elastic core layer is a plurality of strands or a plurality of strips.

A ninth particular aspect includes one or more of aspects 1-8, wherein the elastic core layer is a film, and wherein the plastic core layer is a plurality of strands or a plurality of strips.

In a tenth particular aspect, a disposable absorbent article includes a chassis including an absorbent structure; and a leg gasket attached to the chassis, the leg gasket including an elastic laminate comprising a core layer having a film layer and a strand or strip, wherein the film layer is one of plastic and elastic and the strand or strip is the other of plastic and elastic, and a nonwoven first facing layer affixed to the core layer.

An eleventh particular aspect includes the tenth particular aspect, further including a nonwoven second facing layer affixed to the second surface.

A twelfth particular aspect includes the tenth and/or eleventh aspect, wherein the plastic core layer is a film, and wherein the elastic core layer is a plurality of strands or a plurality of strips.

A thirteenth particular aspect includes one or more of aspects 10-12, wherein the elastic core layer is a film, and wherein the plastic core layer is a plurality of strands or a plurality of strips.

In a fourteenth particular aspect, a method for producing a disposable absorbent article having a leg gasket, the leg gasket including a composite nonwoven elastic web, includes providing an elastic web comprising a core structure having an elastic core layer and a plastic core layer, wherein the elastic web has a first surface and a second surface; stretching the elastic web to less than 100 percent stretch; affixing a fibrous nonwoven web to the first surface of the stretched elastic web to form a composite nonwoven elastic web; relaxing the composite nonwoven elastic web; activating the composite nonwoven elastic web; forming a leg gasket from the composite nonwoven elastic web; and installing the leg gasket in the disposable absorbent article.

A fifteenth particular aspect includes the fourteenth particular aspect, wherein activating includes using a groove rolling process or an intermeshing gears process.

A sixteenth particular aspect includes the fourteenth and/or fifteenth aspect, wherein the elastic core layer is one of a film, a plurality of strands, and a plurality of strips, wherein the plastic core layer is one of a film layer, a plurality of strands, and a plurality of strips.

A seventeenth particular aspect includes one or more of aspects 14-16, wherein the elastic core layer is a film, and wherein the plastic core layer is a plurality of strands or a plurality of strips.

An eighteenth particular aspect includes one or more of aspects 14-17, wherein the plastic core layer is a film, and wherein the elastic core layer is a plurality of strands or a plurality of strips.

A nineteenth particular aspect includes one or more of aspects 14-18, wherein the fibrous nonwoven web is affixed to the elastic web with a thermal, adhesive, ultrasonic, or co-extrusion lamination method.

A twentieth particular aspect includes one or more of aspects 14-19, further including affixing a second fibrous nonwoven web to the second surface.

While the disclosure has been described in detail with respect to the specific aspects thereof, it will be appreciated that those skilled in the art, upon attaining an understanding of the foregoing, can readily conceive of alterations to, variations of, and equivalents to these aspects. Accordingly, the scope of the present disclosure should be assessed as that of the appended claims and any equivalents thereto.

What is claimed is:

1. A leg gasket for a disposable absorbent article, the leg gasket comprising:
a laminate having a core structure with a first surface and a second surface, the core structure comprising an elastic core layer and a plastic core layer, wherein the elastic core layer is one of a film, a plurality of strands, and a plurality of strips, wherein the plastic core layer is one of a film layer, a plurality of strands, and a plurality of strips, and wherein at least one of the elastic and plastic core layers is a film; and a nonwoven first facing layer affixed to the first surface.

2. The leg gasket of claim 1, wherein the plurality of strands is disposed between the film layer and the first facing layer.

3. The leg gasket of claim 1, wherein the first facing layer is affixed to the first surface with adhesive.

4. The leg gasket of claim 1, further comprising a nonwoven second facing layer affixed to the second surface.

5. The leg gasket of claim 1, wherein the nonwoven first facing layer is cellulose-based.

6. The leg gasket of claim 1, wherein the nonwoven first facing layer is polymer-based.

7. The leg gasket of claim 1, wherein the nonwoven first facing layer includes polymer and cellulose.

8. The leg gasket of claim 1, wherein the plastic core layer is a film, and wherein the elastic core layer is a plurality of strands or a plurality of strips.

9. The leg gasket of claim 1, wherein the elastic core layer is a film, and wherein the plastic core layer is a plurality of strands or a plurality of strips.

10. A disposable absorbent article comprising:
a chassis including an absorbent structure; and
a leg gasket attached to the chassis, the leg gasket including an elastic laminate comprising a core layer having a film layer and a strand or strip, wherein the film layer is one of plastic and elastic and the strand or strip is the other of plastic and elastic, and a nonwoven first facing layer affixed to the core layer.

11. The article of claim 10, further comprising a nonwoven second facing layer affixed to the second surface.

12. The article of claim 10, wherein the plastic core layer is a film, and wherein the elastic core layer is a plurality of strands or a plurality of strips.

13. The article of claim 10, wherein the elastic core layer is a film, and wherein the plastic core layer is a plurality of strands or a plurality of strips.

14. A method for producing a disposable absorbent article having a leg gasket, the leg gasket comprising a composite nonwoven elastic web, the method comprising:
providing an elastic web comprising a core structure having an elastic core layer and a plastic core layer, wherein the elastic web has a first surface and a second surface;
stretching the elastic web to less than 100 percent stretch;
affixing a fibrous nonwoven web to the first surface of the stretched elastic web to form a composite nonwoven elastic web;
relaxing the composite nonwoven elastic web;
activating the composite nonwoven elastic web;
forming a leg gasket from the composite nonwoven elastic web; and
installing the leg gasket in the disposable absorbent article.

15. The method of claim 14, wherein activating includes using a groove rolling process or an intermeshing gears process.

16. The method of claim 14, wherein the elastic core layer is one of a film, a plurality of strands, and a plurality of strips, wherein the plastic core layer is one of a film layer, a plurality of strands, and a plurality of strips.

17. The method of claim 16, wherein the elastic core layer is a film, and wherein the plastic core layer is a plurality of strands or a plurality of strips.

18. The method of claim 16, wherein the plastic core layer is a film, and wherein the elastic core layer is a plurality of strands or a plurality of strips.

19. The method of claim 14, wherein the fibrous nonwoven web is affixed to the elastic web with a thermal, adhesive, ultrasonic, or co-extrusion lamination method.

20. The method of claim 14, further comprising affixing a second fibrous nonwoven web to the second surface.
